(19) United States
(12) Patent Application Publication
(10) Pub. No.: US 2015/0126859 A1
(43) Pub. Date: May 7, 2015

(54) GUIDED INCISION PLANNING FOR ENDOSCOPIC MINIMALLY INVASIVE SURGERY

(71) Applicant: KONINKLIJKE PHILIPS N.V., EINDHOVEN (NL)

(72) Inventors: Aleksandra Popovic, New York, NY (US); Haytham Elhawary, New York, NY (US)

(21) Appl. No.: 14/407,539

(22) PCT Filed: Jun. 13, 2013
(86) PCT No.: PCT/IB2013/054854

§ 371 Date: Dec. 12, 2014

Related U.S. Application Data
(60) Provisional application No. 61/660,215, filed on Jun. 15, 2012.

(51) Int. Cl. A61B 19/00; A61B 6/00

(52) U.S. Cl. CPC A61B 19/54; A61B 6/503; A61B 6/5247; A61B 2019/5466; A61B 2019/5483; A61B 2019/5491
USPC 600/426

(57) ABSTRACT

A reference device (166) for surgery includes a substrate (156) forming a matrix of windows (154) configured to be attached to an external portion of a body. The windows provide access location choices from which access to a target area may be determined. A radiopaque marker (150) is integrated with the substrate such that the at least one radiopaque marker is visible in X-ray images. A fixing mechanism (155) is coupled to the substrate to secure the substrate in contact with the body to prevent motion of the substrate relative to the body. Methods and systems using this device are also disclosed.

This disclosure relates to medical instruments and methods and, more particularly, to a reference device employed in locating a target in endoscopic images.

Minimally invasive surgery is performed using elongated instruments inserted into a patient's body through small ports. Placement of ports plays an important role in the outcome of the surgery as instruments positioned in a suboptimal port may not be able to reach all areas of an organ of interest. This is especially a problem in minimally invasive coronary artery bypass surgery where larger incisions between ribs (referred to as a mini-thoracotomy) may be placed to achieve direct access to the heart and specifically to a target artery.

In an ideal case, the position of incision should be exactly above the target artery allowing access without spreading the ribs. However, in many cases, given that there are limited tools to help decide the position of the mini-thoracotomy, the incision can be positioned so that the artery is not directly accessible. In that case, rib retractors or rib lifting devices are needed. Use of such devices may result in postoperative pain, longer recovery times and additional risk of infection.

In accordance with exemplary embodiments of the present invention, a reference device for surgery is disclosed that includes a substrate forming a matrix of windows configured to be attached to an external portion of a body. The windows can provide access location choices from which access to a target area may be determined. A radiopaque marker can be integrated with the substrate such that the at least one radiopaque marker is visible in X-ray images. A fixing mechanism can be coupled to the substrate to secure the substrate in contact with the body to prevent motion of the substrate relative to the body.

For example, the substrate can form a net wherein openings in the net form the windows. The net can include mesh portions and the at least one radiopaque marker can be disposed at an intersection of the mesh portion. The fixing mechanism can include an adhesive and/or a belt or strap. The substrate can include a radiopaque material. Further, the substrate can include one or more indices for identifying the windows, which indices can be visible in an X-ray image in accordance with exemplary embodiments of the present disclosure.

Also in accordance with exemplary embodiments of the present invention, a method for selecting an access location for an anatomical target is disclosed that includes acquiring a first image of an internal anatomical feature; localizing an anatomical target in the first image; applying a reference grid on a body, the reference grid including a substrate forming a matrix of windows configured to be attached to an external portion of the body, the windows providing access location choices from which access to the anatomical target area may be made, the grid including at least one radiopaque marker integrated with the substrate such that the at least one radiopaque marker is visible in X-ray images; acquiring an X-ray image which includes the grid and the anatomical target; registering the first image with the X-ray image; and projecting the grid on the first image to select a grid window for accessing the anatomical target.

For example, acquiring a first image can include acquiring an endoscopic image including the internal anatomical feature and/or acquiring an X-ray image including the internal anatomical feature. Localizing an anatomical target in the first image can include selecting a target blood vessel for a bypass procedure. Projecting the grid on the first image to select a grid window can include selecting a grid window to avoid separating or lifting off of ribs during the bypass procedure. Applying a reference grid on the body can include adhering the reference grid to the body with adhesive. Exemplary embodiments of a method in accordance with the present invention can further comprise accessing the anatomical target through a grid window directly over the anatomical target and/or providing an index on the grid to identify a grid window corresponding to the anatomical target in the X-ray image.

Further, in accordance with exemplary embodiments of the present invention, a system for selecting an access location for an anatomical target is disclosed that includes a first imaging modality configured to generate a first image of an internal anatomical feature and to localize an anatomical target in the first image. A reference grid can be applied on a body. The reference grid can include a substrate forming a matrix of windows configured to be attached to an external portion of the body. The windows can provide access location choices from which access to the anatomical target area may be made. The grid can include at least one radiopaque marker integrated with the substrate such that the at least one radiopaque marker is visible in X-ray images. A second imaging modality can be configured to acquire an X-ray image that includes the grid and the anatomical target. A registering module can be configured to register the first image with the X-ray image. An image processing module can be configured to project the grid on the first image on a display to permit a selection of a best grid window for accessing the anatomical target.

For example, the first image modality can include an endoscope having a camera. The first image can include an X-ray image including the internal anatomical feature. The anatomical target can include a target blood vessel for a bypass procedure. The best grid window can include a grid window that avoids separating or lifting off of ribs during the bypass procedure. The reference grid can include an adhesive structured and configured for adhering the reference grid to the body. It is also possible that best grid window includes a grid window directly over the anatomical target. Exemplary embodiments of a system according to the present disclosure can further comprise an index on the grid to identify a grid window corresponding to the anatomical target in the X-ray image, for example.

These and other objects, features and advantages of the present disclosure will become apparent from the following detailed description of illustrative embodiments thereof, which is to be read in connection with the accompanying drawings.

This disclosure will present in detail the following description of preferred embodiments with reference to the following figures wherein:

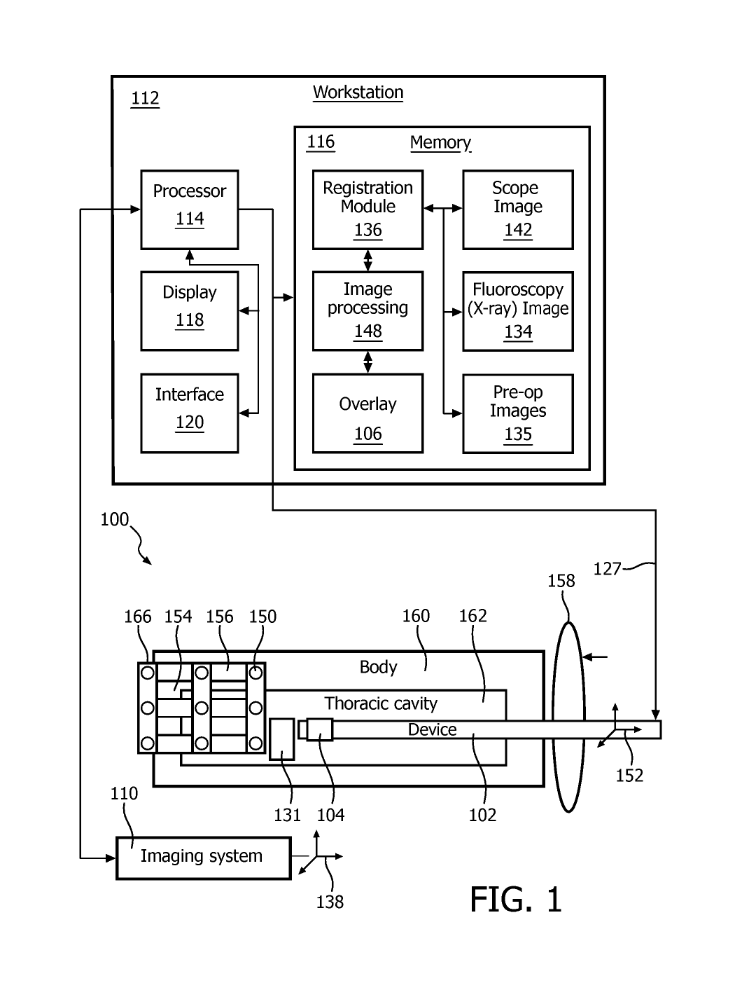

FIG. 1 is a block/flow diagram showing a system for determining an optimal access location for surgery in accordance with one embodiment;

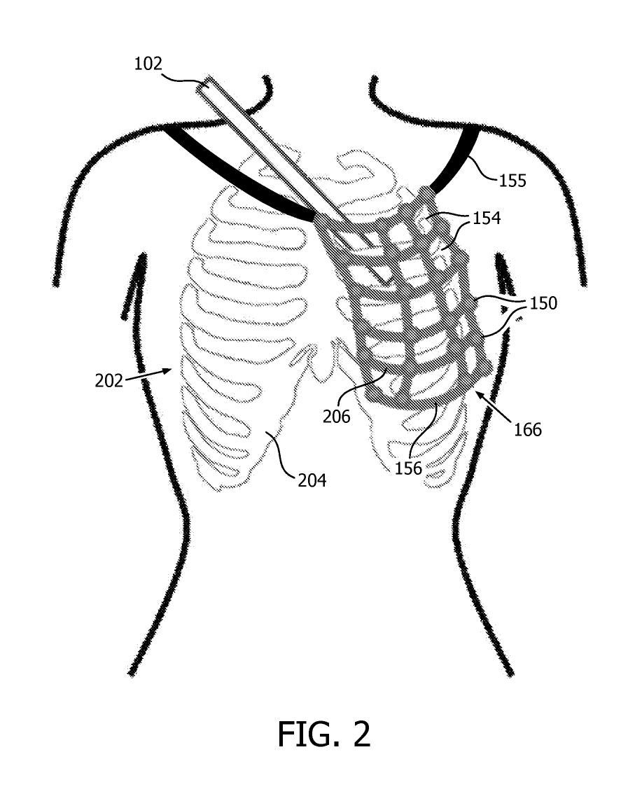

FIG. 2 is a diagram showing a reference grid applied to a thorax in accordance with another illustrative embodiment;

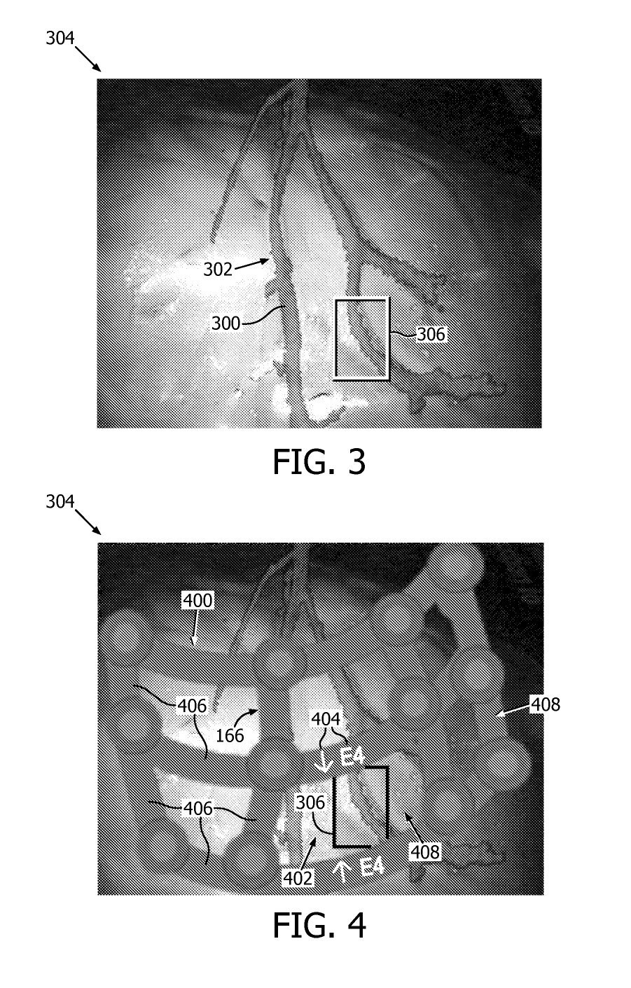

FIG. 3 is an image of an overlay of an arterial tree over an endoscopic image in accordance with one illustrative embodiment; FIG. 4 is the image of FIG. 3 having a grid depicted as a virtual overlay with a target region depicted through one grid window in accordance with an illustrative embodiment;

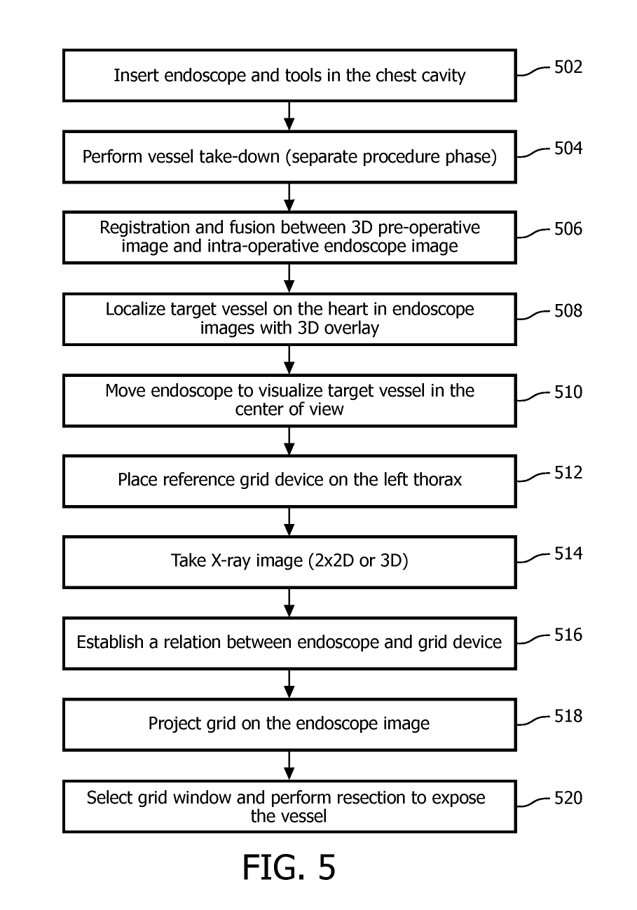

FIG. 5 is a flow diagram showing a method for locating and selecting a best grid window for performing surgery using endoscopic images and X-rays in accordance with an illustrative embodiment;

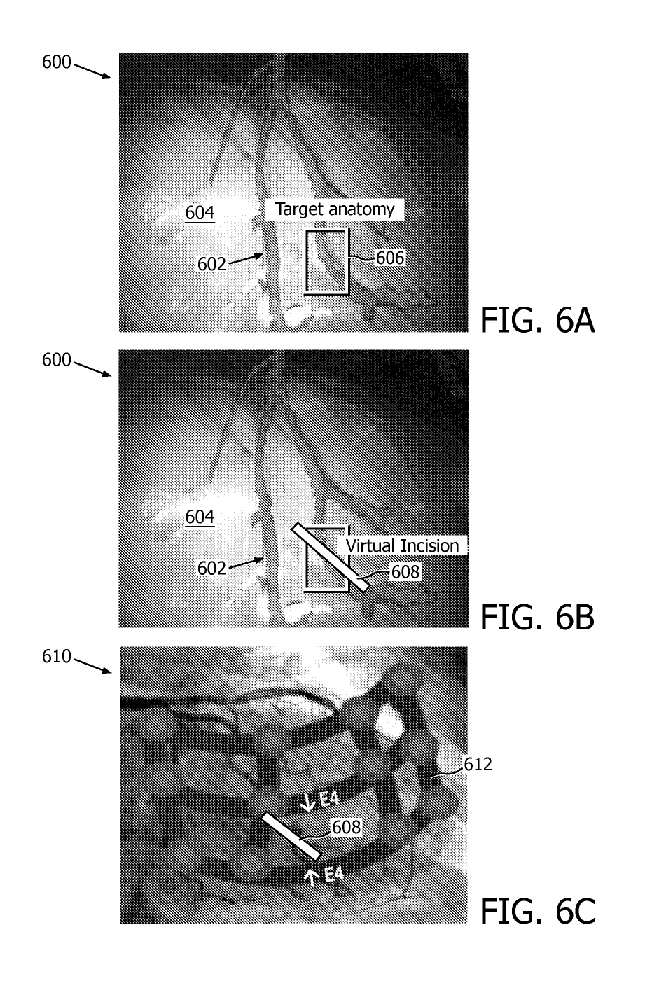

FIG. 6A is an image of an overlay of an arterial tree over an endoscopic image showing a target anatomy in accordance with one illustrative embodiment;

FIG. 6B is an image of the overlay of an arterial tree over the endoscopic image of FIG. 6A showing a virtual incision in accordance with one illustrative embodiment;

FIG. 6C is an X-ray image having a reference grid overlay showing the target anatomy and the virtual incision in accordance with one illustrative embodiment;

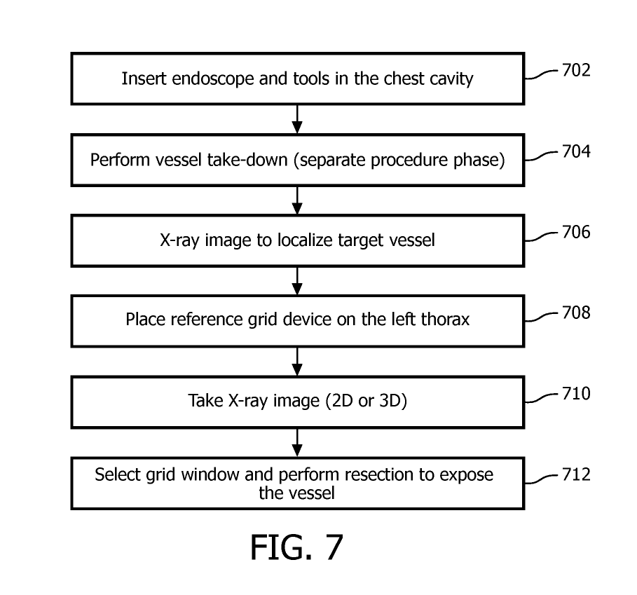

FIG. 7 is a flow diagram showing a method for locating and selecting a best grid window for performing surgery using X-ray images in accordance with an illustrative embodiment;

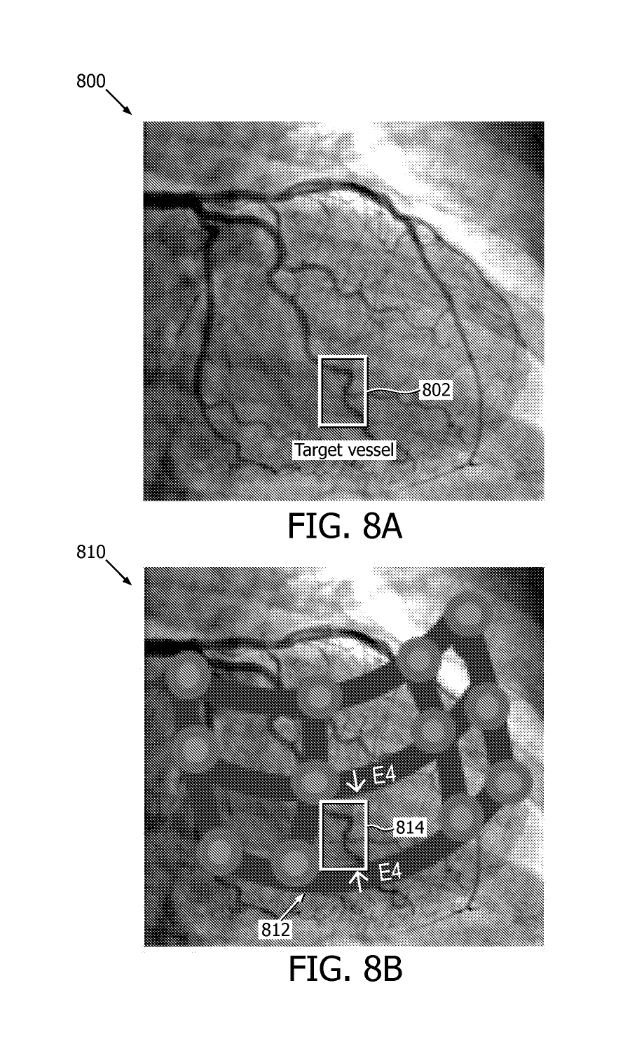

FIG. 8A is an X-ray image of an overlay of an arterial tree showing a target anatomy in accordance with one illustrative embodiment; and

FIG. 8B is an X-ray image having a reference grid overlay showing the target anatomy in accordance with one illustrative embodiment.

In accordance with the present principles, methods for planning and locating a position for an incision for allowing direct access to a target blood vessel within a thoracic cavity (e.g., for a mini-thoracotomy or thoracotomy) without a need to spread or lift the ribs are provided. Intraoperative live incision planning is provided based on intraoperative images (e.g., endoscopy and X-ray) as opposed to using preoperative images. One advantage is that a relative position of ribs and thorax with respect to the heart is established at the time of incision. Preoperative images may have a very different spatial arrangement due to invasive changes introduced intraoperatively (e.g., collapsing the lung, introducing CO₂, etc).

In one embodiment, a patient mounted device is employed to establish a relationship between an imaging modality, e.g., an endoscope and/or X-ray images, and a method to visualize areas on the thorax with respect to the images to precisely plan the incision and remove the need for rib lifting or retraction. The device and methods establish an exact position of mini-thoracotomy with respect to the target vessel (e.g., localize the target vessel in the endoscopy images). The device establishes a reference grid on the thorax with respect to X-ray images. A method to establish a relationship between the endoscope image (target vessel) and the X-ray image (reference grid) is thereby provided, and a most appropriate resection area is identified to access the target vessel.

It should be understood that the present invention will be described in terms of medical instruments; however, the teachings of the present invention are much broader and are applicable to any methods or instruments employed to locate internal targets. In some embodiments, the present principles are employed in accessing or analyzing complex biological or mechanical systems. In particular, the present principles are applicable to internal procedures on biological systems, and procedures in all areas of the body such as the lungs, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W), Blu-Ray™ and DVD.

Referring now to the drawings in which like numerals represent the same or similar elements and initially to FIG. 1, a system 100 for locating a position of a target, such as a blood vessel, is illustratively shown in accordance with one embodiment. System 100 may include a workstation or console 112 from which a procedure is supervised and/or managed. Workstation 112 preferably includes one or more processors 114 and memory 116 for storing programs and applications. Memory 116 may store programs and applications for comparing and registering images. In one embodiment, a registration module 136 is employed to register images using multiple imaging modalities. In a particularly useful embodiment, the imaging modalities may include fluoroscopic (X-ray, computed tomography (CT), etc.) images 134 and/or endoscopic images 142. These images are preferably obtained contemporaneously during the procedure being performed as opposed to, e.g., pre-operative images collected in advance of the procedure, although preoperative images may be employed as well.

A medical device or instrument 102 may include a catheter, a guidewire, a probe, an endoscope, a robot, an electrode, a filter device, a balloon device, other medical component, etc. The medical device 102 includes a camera 104 or other imaging mechanism capable of capturing real-time images internal to a body 160 of a patient. In a particularly useful embodiment, images are collected from inside a thoracic cavity 162 of the body 160. Cabling 127 may be employed to connect the device 102 to the workstation 112 to exchange commands, supply power and transfer data as needed.

The device 102 may be inserted through a port 158, e.g., into the thoracic cavity 162 to locate a target, such as, e.g., a blood vessel 131. For example, the blood vessel 131 may include a blood vessel to be harvested, such as, an internal mammary artery (IMA) or other suitable blood vessel. The port 158 and/or an incision may be employed to access the interior of the thoracic cavity 162 and be employed to insert the device 102 therein. In one embodiment, the device 102 includes an endoscope or a robotically driven endoscope. The camera 104 mounted on or in the device 102 is employed for transmitting internal images to a display 118 and/or to memory 116 for image processing, e.g., registration with other images using the registration module 136 or other image processing or generation using an image processing module 148. The endoscope 102 and/or the camera 104, inserted through the port 158, include a coordinate system 152. Images taken by an imaging system 110 also have their own coordinate system 138. These coordinate systems 138 and 152 can be registered using the registration module 136, and the methods described hereinbelow so that an optimal position for a resection can be determined over a target. The imaging system 110 may include an X-ray system, CT system, etc.

In accordance with one embodiment, a reference grid 166 is placed on the thorax (or other area) and employed with fluoroscopic images. The grid 166 may include a substrate 156, which includes a matrix of one or more radiopaque markers 150 integrated therein. The grid 166 may be attached to the thorax of the body 160 in an area or areas of interest (in cardiac surgery, the grid 166 can be placed on the left side of the patient, on top of the heart). The substrate 156 of the grid 166 forms windows 154, which can be marked with indices, e.g., alpha-numerical markers (numbers, letters, matrix combination of numbers and letters (e.g., B5)) to indicate position in the x-ray image 134.

A relationship between endoscope images 142 and preoperative images 135 and/or intraoperative 3D images 134 (CT angiography or Xper CT angiography) is established using a known method. The endoscope 102 is moved to visualize the target vessel 131, and a target bypass position is centered in the center of the endoscope view. The radiopaque grid 166 is placed on the thorax while the endoscope 102 is maintained in a same position. X-ray imaging is performed to establish a relationship between the endoscope 102 and the grid 166.

As both the endoscope 102 and the grid 166 will be visible in the image 134, a transformation between endoscope coordinate system 152 and the grid 166 can be found. Combining this relationship and the registration performed by registering the endoscope 102 with preoperative/intraoperative 3D images, a relationship between the endoscope image 134 and the grid 166 can be established.

A virtual overlay 106 of the grid 166 may be generated and placed over the endoscope image(s) 142 using the image processing module 148. The virtual overlay 106 over the endoscope image(s) 142 can be displayed on a display 118 using projective geometry and the established relationship described above. The virtual overlay 106 gives an intuitive visualization of the accessibility of the different areas, e.g., on the heart, via different resection ports on the thorax. A resection can be performed such that rib retraction or rib lifting is not necessary, which reduces trauma to the chest, among other things. The grid 166 assists in establishing an exact or optimal position for a mini-thoracotomy, etc. with respect to the target vessel 131 by localizing the target vessel in the endoscopy images 142, establishing the reference grid 166 on the thorax with respect to X-ray images 134, and establishing a relationship between the endoscope image 142 (target vessel) and the X-ray image 134 using the reference grid 166. A most appropriate resection area is then identified to access the target vessel 131.

Workstation 112 includes or is coupled with the display 118 for viewing images of the patient 160. Display 118 may permit a user to interact with the workstation 112 and its components and functions, or any other element within the system 100. This is further facilitated by an interface 120 which may include a keyboard, mouse, a joystick, a haptic device, or any other peripheral or control to permit user feedback from and interaction with the workstation 112.

Referring to FIG. 2, a schematic diagram illustratively shows the grid 166 applied on a thorax 202. A rib cage 204 is indicated for reference. The reference grid 166 on the thorax 202 would appear in X-ray images, as shown. The grid 166 preferably includes an adhesive substrate 156 so that the grid 166 may be reliably secured to the thorax 202 to minimize movement. Adhesives or other mechanisms 155 for securing the grid 166 are contemplated, e.g., belts, straps, tape, hook and loop devices, etc. The substrate 156 may include radiopaque, partially radiopaque or non-radiopaque materials, inks, etc. The grid 166 includes radiopaque markers 150 that are attached to the substrate 156 and applied to the thorax 202 in the area of interest. For example, for cardiac surgery, the grid 166 can be placed over a left side of the chest on top of the heart. The grid 166 includes windows 154 which can be varied in size and shape depending on the application. The grid 166 may be marked with radiopaque or X-ray visible indexes 206. The indexes 206 may include numbers, letters, matrix combinations of numbers and letters, etc. to indicate different locations on the grid 166.

It should be understood that while the grid 166 is depicted as a net-like shape, the shape of the grid may include concentric circles, a single line with a plurality of parallel intersecting lines, concentric rectangles, etc. The windows 154 may be pre-shaped or located to provide the best opportunity for accessing an anatomical target. For example, the grid 166 may include location points that can be applied over external anatomical features (e.g., ribs, the sternum, etc.) to better position the windows 154 at optimal locations. The windows 154 represent access locations where a high probability of clear access can be obtained, which is verified directly using the virtual overlay image.

In a first step of an illustrative procedure, a relationship between an endoscope video stream as collected by the endoscope 102 and preoperative or intraoperative 3D images is established. Endoscopic registration to preoperative images may employ known techniques. The endoscope 102 is then moved to a position where a target vessel and a target bypass position can be visualized (e.g., in the center of the endoscope view). In a second step, the radiopaque grid 166 is placed on the thorax while the endoscope 102 is maintained in the same position. In the third step, X-ray imaging is performed to establish a relationship between the endoscope 102 and the grid 166. Both endoscope 102 and the grid 166 will be visible in the image. Therefore, a transformation between an endoscope coordinate system and the grid 166 can be found. Combining this relationship and the registration performed in the first step, a relationship between endoscope image and the grid 166 can be established. In a fourth step, a virtual overlay of the grid in endoscope images can be displayed using projective geometry and the established relationship.

Referring to FIG. 3, an overlay image 300 shows an arterial tree 302 overlaid on an endoscope image 304 of a heart. A selected area (rectangle) 306 has been selected for bypass. This overlay image 300 may be generated in the first step, for example, when the relationship between an endoscope video stream as collected by the endoscope 102 (FIG. 1) and preoperative or intraoperative 3D images is established.

Referring to FIG. 4, the image 304 of FIG. 3 is shown having a virtual overlay 400 of a segment of the grid 166 on the endoscope image 304 to visualize a resection area 402 which includes the selected area 306. Additional markings or indices 404 on the grid 166 can be employed to directly locate an appropriate area. In one example, a mini-thoracotomy may be desired; the index “E4” with arrows may be employed to indicate the selected area 306. It should be understood that portions 406 of the grid 166 may be customizable to line up with anatomical features. For example, the grid 166 may include a portion or portions 406 that follow the locations of the ribs, blood vessels, etc. so that these anatomical features can be made visible in the endoscopic image using the virtual overlay 400. The virtual overlay 400 gives an intuitive visualization of the accessibility of the different areas on the heart via different resection ports on the thorax corresponding with grid windows 408. Thus, a resection can be performed, in this example, such that rib refraction or rib lifting is not necessary thereby reducing trauma to the chest.

Referring to FIG. 5, a method for performing a heart bypass procedure in accordance one illustrative embodiment is depicted. In block 502, an endoscope and tools for the procedure are inserted into a chest cavity. In block 504, a vessel take-down is performed. This includes identifying and removing a suitable blood vessel to be employed in replacing damaged or blocked blood vessels in the heart. In block 506, registration is made between 3D preoperative images of the chest cavity and the endoscopic (intra-operative) images collected of the chest cavity. The registered images are preferably fused into a single image or image stream. This may include a virtual overly of blood vessels onto the endoscopic images. In block 508, once the overlay (in 3D) is provided, a target vessel is localized on the heart in the endoscopic images. In block 510, the endoscope is moved by hand or by robot to center the localized blood vessel on the heart. In block 512, a reference grid, as described above, is placed over the heart (left thorax).

In block 514, an X-ray image is taken with the reference grid and preferably the endoscope position in view. The X-ray image may be a 2×2D or 3D X-ray. In block 516, a relationship between the endoscope and the reference grid is established. In block 518, the reference grid is projected onto the endoscope image. In block 520, a window is selected in the reference grid that avoids ribs or other regions of difficulty and a resection is performed to access the target area.

Referring to FIGS. 6A-6C, illustrative images showing points of interest of the workflow described with respect to FIG. 5 are depicted. In FIG. 6A, an overlay image 602 on a heart 604 including a target anatomy 606 is provided on an endoscope image 600. In FIG. 6B, a “virtual incision” 608 may be drawn by a physician onto the endoscope image 600. The physician can draw, with a mouse or other input device on the endoscope image 600 (e.g., on a display screen), the position and orientation of the incision 608 he or she would like to make. In FIG. 6C, the reference grid 166 is projected as placed on the thorax as an overlay image 612, and a relationship (e.g., transformation) is established between the x-ray image coordinate frame and the endoscope coordinate frame. Once this relationship has been established, the virtual incision 608 that was drawn over the endoscope image can be overlaid on the previously acquired x-ray images 610. The previously acquired x-ray images 610 include the overlay 612 of the reference grid 166, and the part of the grid 166 where the incision will give the best access to the target anatomy can be selected.

Referring to FIG. 7, another illustrative embodiment is described. In block 702, an endoscope and tools for the procedure are inserted into a chest cavity. In block 704, a vessel take-down is performed by a separate procedure. In block 706, an X-ray image is taken of the chest cavity to localize a target vessel. This is depicted in FIG. 8A where a target vessel 802 is shown in an X-ray image 800. In block 708, a reference grid, as described above, is placed over the heart (left thorax). In block 710, an X-ray image is taken with the reference grid and preferably the endoscope position in view. The X-ray image may be a 2D or 3D X-ray image. In block 712, the reference grid is projected onto the X-ray image taken in block 710 and employed to select a window in the reference grid that avoids ribs or other regions of difficulty. A resection can be performed to access or expose the target area. FIG. 8B shows an X-ray image 810 having a reference grid overlay 812 and target area 814 projected onto the X-ray image 810.

In the case of cardiac bypass grafting, the target coronary artery can often be identified in the X-ray image by a narrowing of the vessel (See FIG. 8A). If this is the case, the image which includes the radiopaque grid (FIG. 8B) can be used to select the part of the grid where the incision will give the best access to the target anatomy. In the case where the target vessel cannot be seen in the x-ray image, registration and image fusion between the x-ray and some other 3D pre-operative image (such as CT-angiography), with methods known in art, can help localize the vessel in the x-ray image.

The present principles may be employed for different applications including endoscopically-guided minimally invasive surgeries or procedures. These procedures and surgeries are not limiting, and the present principles may be employed in, e.g., cardiac surgery, minimally invasive coronary artery bypass grafting, atrial septal defect closure, valve repair/replacement, laparoscopic surgery, hysterectomy, prostatectomy, gall bladder surgery, natural orifice transluminal surgery (NOTES), pulmonary/bronchoscopic surgery, neurosurgical interventions, video assisted thoracic surgery, etc.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for guided incision planning for endoscopic minimally invasive surgery (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A reference device for surgery, comprising:
a substrate forming a matrix of windows configured to be attached to an external portion of a body, the windows providing access location choices from which access to a target area may be determined, wherein the substrate forms a net and openings in the net form the windows;
at least one discrete radiopaque marker attached to a top surface of the substrate such that the at least one radiopaque marker is visible in X-ray images; and
a fixing mechanism coupled to the substrate to secure the substrate in contact with the body to prevent motion of the substrate relative to the body.

2. (canceled)

3. The reference device as recited in claim 1, wherein the net includes mesh portions and the at least one radiopaque marker is disposed at an intersection of the mesh portion.

4. The reference device as recited in claim 1, wherein the fixing mechanism includes an adhesive.

5. The reference device as recited in claim 1, wherein the fixing mechanism includes a belt or strap.

6. The reference device as recited in claim 1, wherein the substrate includes a radiopaque material.

7. The reference device as recited in claim 1, wherein the substrate includes one or more indices for identifying the windows.

8. The reference device as recited in claim 7, wherein the one or more indices are visible in an X-ray image.

9. A system for selecting an access location for an anatomical target, comprising:
a first imaging modality configured to generate a first image of an internal anatomical feature and to localize an anatomical target in the first image;
a reference grid applied on a body, the reference grid including a substrate forming a matrix of windows configured to be attached to an external portion of the body, the windows providing access location choices from which access to the anatomical target area may be made, the grid including at least one radiopaque marker integrated with the substrate such that the at least one radiopaque marker is visible in X-ray images;
a second imaging modality configured to acquire an X-ray image that includes the grid and the anatomical target;
a registering module configured to register the first image with the X-ray image; and
an image processing module configured to project the grid on the first image on a display to permit a selection of a best grid window for accessing the anatomical target.

10. The system as recited in claim 9, wherein the first image modality includes an endoscope having a camera.

11. The system as recited in claim 9, wherein the first image includes an X-ray image including the internal anatomical feature.

12. The system as recited in claim 9, wherein the anatomical target includes a target blood vessel for a bypass procedure, and wherein the best grid window includes a grid window that avoids separating or lifting off of ribs during the bypass procedure.

13. The system as recited in claim 9, wherein reference grid includes an adhesive for adhering the reference grid to the body.

14. The system as recited in claim 9, further comprising an index on the grid to identify a grid window corresponding to the anatomical target in the X-ray image, wherein the best grid window includes a grid window directly over the anatomical target.

15. A method for selecting an access location for an anatomical target, comprising:
acquiring a first image of an internal anatomical feature;
localizing an anatomical target in the first image;
applying a reference grid on a body, the reference grid including a substrate forming a matrix of windows configured to be attached to an external portion of the body, the windows providing access location choices from which access to the anatomical target area may be made, the grid including at least one radiopaque marker integrated with the substrate such that the at least one radiopaque marker is visible in X-ray images;
acquiring an X-ray image which includes the grid and the anatomical target;
registering the first image with the X-ray image; and
projecting the grid on the first image to select a grid window for accessing the anatomical target.
